(19) United States
(12) Patent Application Publication
(10) Pub. No.: US 2022/0047920 A1
(43) Pub. Date: Feb. 17, 2022

(54) SYSTEMS AND METHODS FOR PERSONALIZED FITNESS ASSESSMENTS AND WORKOUT ROUTINES

(71) Applicant: True Adherence, Inc., Salt Lake City, UT (US)

(72) Inventors: Kyle Poulin, Salt Lake City, UT (US); Chris Bright, Salt Lake City, UT (US)

(21) Appl. No.: 17/401,308

(22) Filed: Aug. 12, 2021

Related U.S. Application Data
(60) Provisional application No. 63/064,471, filed on Aug. 12, 2020.

(51) Int. Cl. A63B 24/00; G06K 9/00; A63B 22/02; G16H 20/30

(52) U.S. Cl. CPC A63B 24/0075; G06K 9/00348; G06K 9/00671; A63B 2024/0068; A63B 24/0062; G16H 20/30; A63B 2220/806; A63B 22/02

(57) ABSTRACT

A system to generate an adapted workout plan includes an input reception device to receive input data from a user and a workout routine modeling engine coupled to the input reception device. The workout routine modeling engine is configured to analyze the input data from a user and generate an adapted workout routine for the user based on the input data.

### CROSS-REFERENCE TO RELATED APPLICATIONS

This application claims the benefit of U.S. Provisional Application No. 63/064,471, filed on Aug. 12, 2020, which is incorporated by reference herein in its entirety.

### BACKGROUND

It is important for athletes and individuals to exercise and train properly, not only to maintain and improve their physical well-being but to avoid over-stressing or injuring specific muscles, joints, or other parts of the body being worked. As such, it is common to retain personal human trainers to assist and guide in a personal workout. But personal trainers are not always available and may not have all relevant information available that may be helpful or even essential for a more fully customized, personal fitness, training, or conditioning exercise workout program. A proper program is complicated to generate. Components of resistance training, cardiovascular endurance, joint mobility, muscle flexibility, athlete agility and recovery must all be incorporated to optimize the progress of the individual.

Embodiments of the present invention allow for the optimization of workouts without a personal human trainer being necessary. Other aspects and advantages of embodiments of the present invention will become apparent from the following detailed description, taken in conjunction with the accompanying drawings, illustrated by way of example of the principles of the invention.

### SUMMARY

Embodiments of a system are described. A system to generate an adapted workout plan includes an input reception device to receive input data from a user and a workout routine modeling engine coupled to the input reception device. The workout routine modeling engine is configured to analyze the input data from a user and generate an adapted workout routine for the user based on the input data. Other embodiments of the system are also described. The preceding subject matter of this paragraph characterizes example 1 of the present disclosure.

The input reception device includes a camera to record a workout routine of the user. The preceding subject matter of this paragraph characterizes example 2 of the present disclosure, wherein example 2 also includes the subject matter according to example 1, above.

The workout routine modeling engine is configured to analyze parameters of the workout routine of the use. The preceding subject matter of this paragraph characterizes example 3 of the present disclosure, wherein example 3 also includes the subject matter according to any one of examples 1-2, above.

The input reception device is configured to receive manually entered data from the user. The preceding subject matter of this paragraph characterizes example 4 of the present disclosure, wherein example 4 also includes the subject matter according to any one of examples 1-3, above.

The workout routine modeling engine includes an AI algorithm that is configured to analyze the input data. The preceding subject matter of this paragraph characterizes example 5 of the present disclosure, wherein example 5 also includes the subject matter according to any one of examples 1-4, above.

The workout routine modeling engine develops a reference movement profile, wherein the reference movement profile comprises expected three-dimensional positions and movements associated with the user. The preceding subject matter of this paragraph characterizes example 6 of the present disclosure, wherein example 6 also includes the subject matter according to any one of examples 1-5, above.

The workout routine modeling engine is configured to determine whether the input data includes deviations from the reference movement profile. The preceding subject matter of this paragraph characterizes example 7 of the present disclosure, wherein example 7 also includes the subject matter according to any one of examples 1-6, above.

The workout routine modeling engine is configured to deviate from the adapted workout routine for the user when the deviations are above a threshold. The preceding subject matter of this paragraph characterizes example 8 of the present disclosure, wherein example 8 also includes the subject matter according to any one of examples 1-7, above.

The workout routine modeling engine is configured to update the reference movement profile with the deviations for subsequent workouts. The preceding subject matter of this paragraph characterizes example 9 of the present disclosure, wherein example 9 also includes the subject matter according to any one of examples 1-8, above.

The workout routine modeling engine is further configured to develop a form score, wherein the form score quantifies conformity of the input data to the reference movement profile. The preceding subject matter of this paragraph characterizes example 10 of the present disclosure, wherein example 10 also includes the subject matter according to any one of examples 1-9, above.

The workout routine modeling engine is configured to average a form score of each repetition of an exercise to determine a form score of a set of repetitions. The preceding subject matter of this paragraph characterizes example 11 of the present disclosure, wherein example 11 also includes the subject matter according to any one of examples 1-10, above.

A method for generating an adapted workout plan includes analyzing input data of a user received by an input reception device, wherein the input data comprises workout information for the user and utilizing a workout routine modeling engine coupled to the input reception device to generate an adapted workout routine for the user based on the input data. The preceding subject matter of this paragraph characterizes example 12 of the present disclosure.

The method includes monitoring a user in at least one workout routine with the input reception device. The preceding subject matter of this paragraph characterizes example 13 of the present disclosure, wherein example 13 also includes the subject matter according to example 12, above.

The method includes receiving the input data through manual input by the user. The preceding subject matter of this paragraph characterizes example 14 of the present disclosure, wherein example 14 also includes the subject matter according to any one of examples 12-13, above.

The workout routine modeling engine utilizes an AI algorithm that is configured to analyze the input data and determine the adapted workout routine for the user based on the input data. The preceding subject matter of this paragraph characterizes example 15 of the present disclosure, wherein example 15 also includes the subject matter according to any one of examples 12-14, above.

The method includes developing a reference movement profile, wherein the reference movement profile comprises expected three-dimensional positions and movements associated with the user. The preceding subject matter of this paragraph characterizes example 16 of the present disclosure, wherein example 16 also includes the subject matter according to any one of examples 12-15, above

The method includes determining whether the input data includes deviations from the reference movement profile. The preceding subject matter of this paragraph characterizes example 17 of the present disclosure, wherein example 17 also includes the subject matter according to any one of examples 12-16, above.

The method includes deviating from the adapted workout routine for the user when the deviations are above a threshold. The preceding subject matter of this paragraph characterizes example 18 of the present disclosure, wherein example 18 also includes the subject matter according to any one of examples 12-17, above

The method includes updating the reference movement profile with the deviations for subsequent workouts. The preceding subject matter of this paragraph characterizes example 19 of the present disclosure, wherein example 19 also includes the subject matter according to any one of examples 12-18, above

The method includes developing a form score, wherein the form score quantifies conformity of the input data to the reference movement profile. The method includes averaging a form score of each repetition of an exercise to determine a form score of a set of repetitions. The preceding subject matter of this paragraph characterizes example 20 of the present disclosure, wherein example 20 also includes the subject matter according to any one of examples 12-19, above.

Other aspects and advantages of embodiments of the present invention will become apparent from the following detailed description, taken in conjunction with the accompanying drawings, illustrated by way of example of the principles of the invention.

### BRIEF DESCRIPTION OF THE DRAWINGS

In order that the advantages of the subject matter may be more readily understood, a more particular description of the subject matter briefly described above will be rendered by reference to specific embodiments that are illustrated in the appended drawings. Understanding that these drawings depict only typical embodiments of the subject matter and are not therefore to be considered to be limiting of its scope, the subject matter will be described and explained with additional specificity and detail through the use of the drawings, in which:

FIG. 1 depicts a schematic diagram of a system to generate an adapted workout plan according to one or more embodiments of the present disclosure;

FIG. 2 depicts a schematic diagram of a system to generate an adapted workout plan according to one or more embodiments of the present disclosure;

FIG. 3 is an example of the primary hardware components used to run the system according to one or more embodiments of the present disclosure;

FIG. 4 illustrates the various places which the platform may be accessed along with the types of activities observed according to one or more embodiments of the present disclosure;

FIG. 5 shows a cardio analysis tool according to one or more embodiments of the present disclosure;

FIG. 6A shows an example of a user interacting with an interface of a kiosk that is running a resistance tool according to one or more embodiments of the present disclosure;

FIG. 6B shows an example of a user interacting with an interface of a kiosk that is running a resistance tool according to one or more embodiments of the present disclosure;

FIG. 6C shows an example of a user interacting with an interface of a kiosk that is running a resistance tool according to one or more embodiments of the present disclosure;

FIG. 7 shows a rehabilitation assessment tool associated with the platform according to one or more embodiments of the present disclosure;

FIG. 8 shows a web portal or phone application that allows users to have access to their data according to one or more embodiments of the present disclosure;

FIG. 9 shows a professional portal where therapists or personal trainers may access the data of clients with permission according to one or more embodiments of the present disclosure;

FIG. 10 illustrates how the system modifies workouts based on observed information from the various inputs according to one or more embodiments of the present disclosure;

FIG. 11 depicts a schematic diagram of a system according to one or more embodiments of the present disclosure; and

FIG. 12 depicts a flow chart diagram of a method according to one or more embodiments of the present disclosure.

Throughout the description, similar reference numbers may be used to identify similar elements.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment.

This disclosure describes an example of a smart personal training platform or system. In some embodiments, the system automatically generates personalized wholistic workout routines to achieve user goals. In some embodiments, the system is configured to adjust workout routines based on input data.

The system and platform enables user to login and create a personal profile unique to the user. The system may then automatically guide the user through a series of assessments (mobility, resistance, agility, and cardiovascular in nature). In some embodiments, the system is configured to create a dot map of the user. This may be created with an infrared camera or other depth sensing device. The dot map may be a “skeleton” of the individual, taking measurements, and creating and providing visual feedback including biomechanical indications of the user.

In some embodiments, the system may also receive input from the user direction. The user may complete a behavioral assessment to outline goals, pertinent medical history, availability for exercise, and desired reminders. From the assessments, a wholistic workout program may be created that incorporates resistance, cardiovascular, mobility, agility, and flexibility training with suggested dates and times to perform them. The user then begins the workout program.

While engaging in the various workout routines, the system may be configured to provide the user with biomechanically appropriate feedback, based on the activity being completed. In some embodiments, the system may coach the user regarding how to improve intra-workout via visual and auditory cues. Throughout the workout, various data points are captured to guide future performance.

An example platform would include features such as rest timer, summary feedback and statistics provided at the end of each set and at the end of an entire workout, a virtual trainer option, automatic adjustment of the exercise workout routine based on data collected, social media features, gamification features, exercise calibration features, tutorials, distance achieved, peak heart rate, heart rate variability, comparative performance to previous workouts and other information.

The system or platform may rely on hardware and software components in some embodiments. The system may include various components that are described herein in more detail. The system may include only some or all of the components described herein in differing embodiments.

The system may include a decision-making engine which is configured to perform many of the steps outlined herein. The decision-making engine may be part or wholly software. The system also includes hardware in some embodiments. The hardware is predominantly used to capture inputs that enables the software to generate feedback and suggestions. The hardware may include sensors, cameras, computing devices, and the like that are able to receive data, analyze data, and output data.

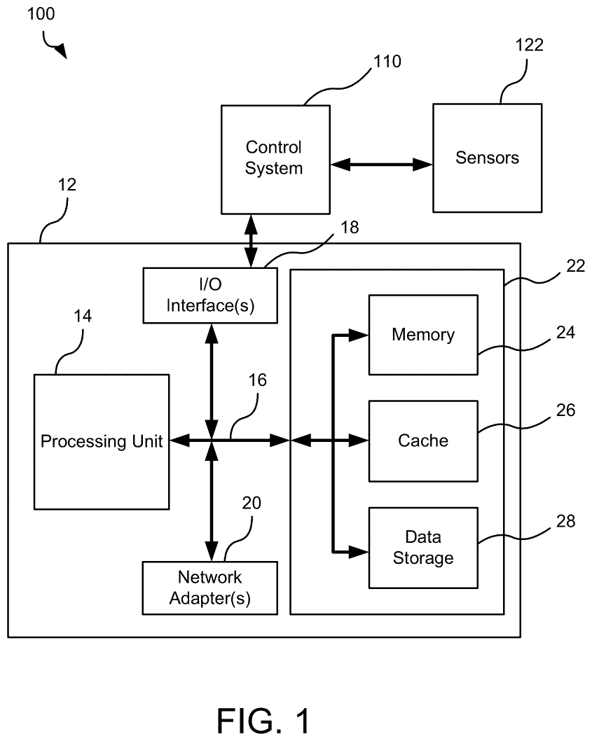

Referring to FIG. 1, a schematic diagram of a workout system 100 is shown. Although the workout system 100 is shown and described with certain components and functionality, other embodiments of the workout system 100 may include fewer or more components to implement less or more functionality.

In some embodiments, aspects of the workout system 100 are implemented via a networked system or a computer system 12 or its component parts. The illustrated computer system 12 may include, but is not limited to, one or more processing arrangements, for example including processors or processing units 14, a communication bus 16, one or more input/output (I/O) adapters 18, one or more network adapters 20, and a system memory 22.

In one embodiment, the system memory 22 includes computer system readable media in the form of volatile memory, such as random-access memory (RAM) 24 and/or cache memory 26. The system memory 22 may further include other removable/non-removable, volatile/non-volatile computer system storage media 28 In such instances, each can be connected to the bus 16 by one or more data media interfaces. The memory 22 may include at least one program product having a set (e.g., at least one) of program modules that are configured to carry out the functions of proposed embodiments. For instance, the memory 22 may include a computer program product having program executable by the processing unit 14 to perform processes described herein. Programs and/or utilities having a set (at least one) of program modules may be stored in the memory 22. Program modules generally carry out the functions and/or methodologies described herein.

The computer system 12 may also communicate with one or more external devices such as a keyboard, a display, sensors 122, cameras, apps, or other external devices, including but not limited to a control system 110. Also, the computer system 12 can communicate with one or more networks such as a local area network (LAN), a general wide area network (WAN), and/or a public network (e.g., the Internet) via network adapter 20.

In the context of the present application, where embodiments of the present invention constitute a method, it should be understood that such a method is a process for execution by a computer, i.e. is a computer-implementable method. The steps of the method therefore reflect various parts of a computer program, e.g., parts of one or more algorithms. Embodiments of the present invention may be a system, a method, and/or a computer program product. The computer program product may include a computer readable storage medium (or media) having computer readable program instructions thereon for causing a processor to carry out aspects of the present invention.

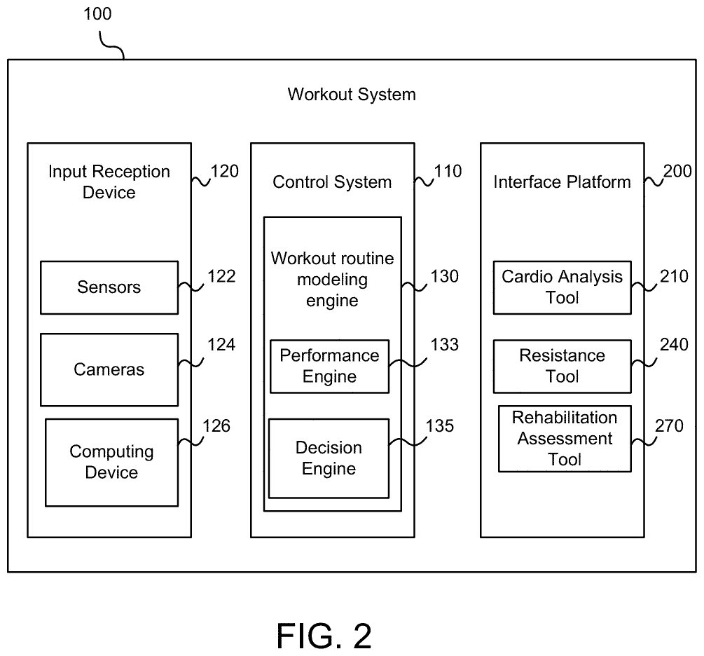

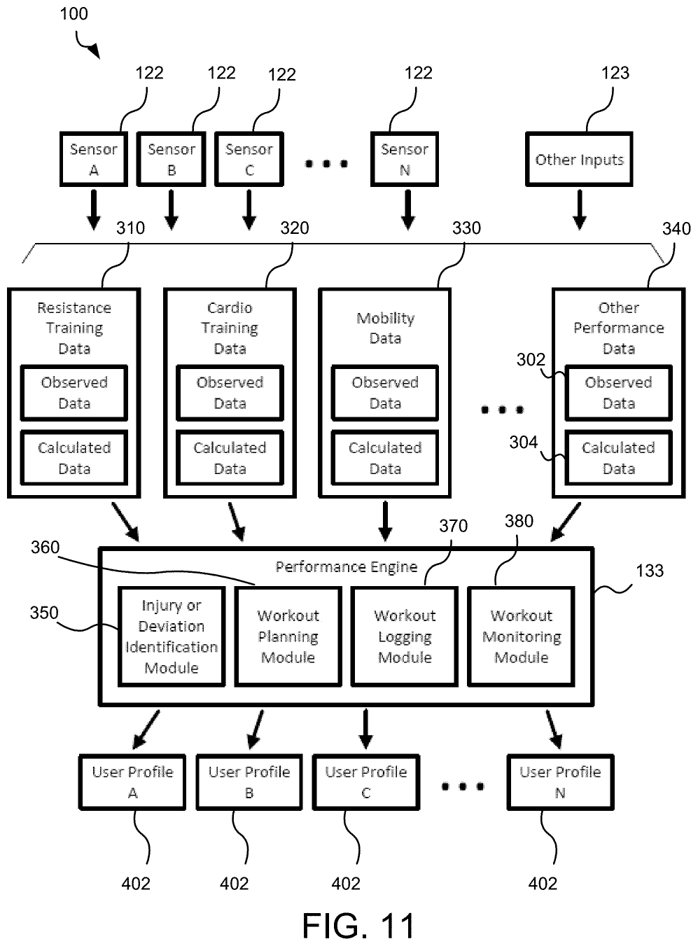

Referring to FIG. 1 again, the control system 110 interacts with and receives signals from sensors 80. The control system 110 is further configured to control the system 100 and its function. In some embodiments, more than one control system 110 may control the various components of the system 100 (this applies to FIGS. 2 and 11 as well as the other figures within this disclosure).

Referring to FIG. 2, a schematic diagram of another embodiment of a workout system 100 is shown. The embodiment depicted in FIG. 2 may include any or all the features described in conjunction with FIG. 1. Additionally, all the features described in conjunction with FIG. 2 may be included with the embodiments associated with FIG. 1.

The workout system 100 includes a control system 110, an input reception device 120, and an interface platform 200. The control system 110 includes a workout routine modeling engine 130 which may include a performance engine 133 and a decision engine 135. As discussed above, the control system 110 is configured to control and operate the system 100 and interface with a input reception device 120 and an interface platform 200, in some embodiments. The workout system 100 also includes software and hardware to perform the functions and method steps described herein. The software may include various modules that are designed to store and run algorithms and input data or other information. The software may also be configured to execute various algorithms including a deviation identification algorithm associated with a deviation identification module, a workout planning algorithm associated with a workout planning module, a workout logging algorithm associated with a workout logging module, and a workout monitoring algorithm associated with a workout monitoring module. See discussion of FIG. 11 regarding such example modules.

The illustrated embodiment of a workout system 100 also includes an interface platform 200. In the illustrated embodiment, the interface platform 200 includes cardio analysis tool 210, a resistance tool 240, and a rehabilitation assessment tool 270. Although the interface platform 200 is shown and described with certain components and functionality, other embodiments of the interface platform 200 may include fewer or more components to implement less or more functionality. These components are described in more detail in conjunction with the remaining Figures.

The illustrated embodiment of a workout system 100 also includes an input reception device 120. In the illustrated embodiment, the input reception device 120 includes sensors 122, cameras 124, and a computing device 126. Although the input reception device 120 is shown and described with certain components and functionality, other embodiments of the input reception device 120 may include fewer or more components to implement less or more functionality. These components are described in more detail in conjunction with the remaining Figures. The computing device 126 may be a mobile phone, a computer such as a laptop or desktop computer, a human-sized workout kiosk, or the like.

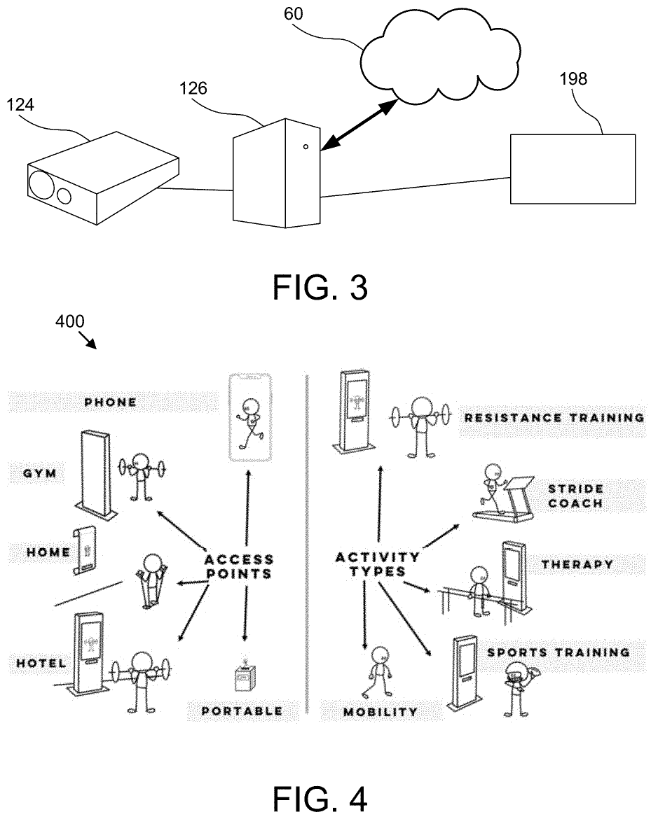

FIG. 3 is an example of some primary hardware components that may be used to run the system 100. Depicted are a depth sensing camera 124. In some embodiments, camera 124 may include various technologies for recording or observing a user, whether infrared (IR) or Light Detection and Ranging (LiDAR), or 2D-video images extrapolated to 3D visualizations. The camera 124 along with other sensors provide key inputs to the system 100. In some embodiments, it is desirable to have depth sensing capability. The data captured by the camera 124 is processed by the software that is either hosted on a local computer 126 or other computing device or via the cloud 60 to a remote computing device. This information may be output to a screen or projector or display 198 that is attached to the system 100.

Depending on the components used, various housings and configurations may be used to optimize the observed inputs. For example, while resistance training, all the components may be combined in a singular kiosk. While running on a treadmill, the camera 124 may attach to or be suspended by the equipment. Wires could be led to an onsite computer 126 or to an internet router to allow for the processing of the inputs remotely. There would then be an attached screen where the coaching and feedback would be displayed. Again, this screen could be on the treadmill or suspended nearby.

Secondary input devices may include touchscreens on a kiosk or treadmill to enable the user to input information. Microphones may be included to allow the user to give audio feedback for the software to consider. Biometric data is also gathered with heart rate monitors.

To enhance the accuracy of the depth sensing cameras, lighting may be incorporated into the various housing. If just a camera is used, with no local computer, the lights may be attached to the camera itself or directly incorporated into the workout area of the user.

With enhanced accuracy, the depth sensing cameras also facilitate object recognition. This will allow the platform to automatically identify various weight types and sizes being used for resistance training, objects used to enhance stretching/mobility, and bands used for agility training.

The platform automatically modifies routines based on the variety of observed inputs from the various exercise types. Should a user struggle to fully complete a resistance exercise (e.g., barbell back squat), the next set of lifts would be modified to ensure the user could complete the lift. The modification may change the amount of rest time between sets, the weight being lifted, or the type of lift being done. Information between various exercise types (resistance, cardiovascular, mobility, agility and flexibility.) For example, should a user have a “peak” performance day on cardiovascular work (e.g., ran a marathon) the subsequent workouts would be modified to optimize recovery. Thus, a “heavy” resistance day would be moved to a later date and mobility/flexibility trainings would be prioritized with an emphasis on the areas most affected (e.g., legs, shoulders and trunk.)

Similarly, should a user be preparing for a future event, (e.g., a strongman competition) the platform would develop progressions toward this event. However, should observed performance falter (e.g., drops in velocity, volume lifted, time under tension) persist, the system will modify programming to ensure continued progression.

If an injury has occurred to the individual, the system will re-assess the capabilities of the user. From there, new workouts will be generated that adapt for the injury and optimize the rehabilitation of the individual. The platform may be incorporated into a physical therapy practice to allow certified therapists to work with the patient. The data the platform gathers would be used to provide suggestions to the therapist and the user. If the user has an at home version, data would be observed for the adherence to the workout regimen provided by the Therapist.

FIG. 4 illustrates the various places which the platform may be accessed along with the types of activities observed. As suggested above, users may obtain information from rehabilitation through to performance facilities. Illustrated are a gym, a home, and a hotel. Each of these locations may communicate through the cloud 60 to aggregate information and data for input.

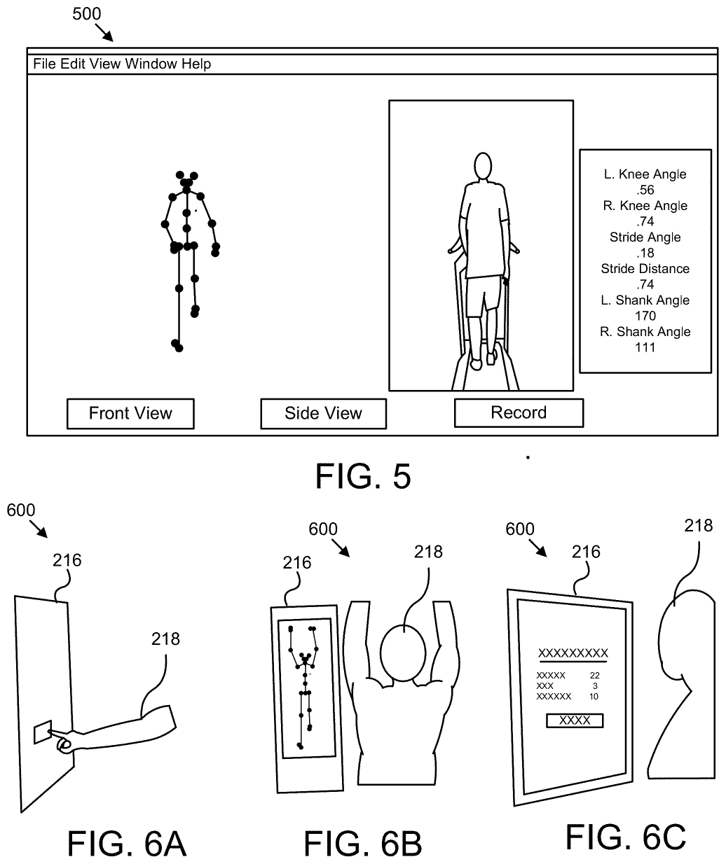

FIG. 5 shows an example of a schematic 500 of a cardio analysis tool 210. The camera may be positioned in front, to the side, or behind an individual. The camera height position may vary based on the design of the treadmill to ensure the whole person is in the viewing screen. The processed video is extrapolated to a 3D dot map that has the ability to be viewed from any 360-degree spherical point. Per the image, various angles may be observed including, but not limited to, stride angle, shank angle, dorsiflexion, plantar flexion, elbow flexion, knee flexion. Various velocity, pace, workout time, movements patterns, and other statistics are also observed through this method.

FIG. 6A shows an example 600 of a user 218 interacting with an interface of a kiosk 216 that is running a resistance tool 240. FIG. 6B shows an example 600 of a user 218 interacting with an interface of a kiosk 216 that is running a resistance tool 240. This example shows a dot map of an individual that, like the stride tool, may use a camera in front, behind, or to the side of the individual. The dot map may be rotated as well. Here we observe angles of the body, but also performance metrics of resistance training such as form score, time under tension, velocity of various joints, velocity of a workout apparatus (barbell, dumbbell kettlebell, etc.), total workout time, movement patterns, movement variance, and other statistics pertinent to resistance training. FIG. 6C shows an example 600 of a user 218 interacting with an interface of a kiosk 216 that is running a resistance tool 240.

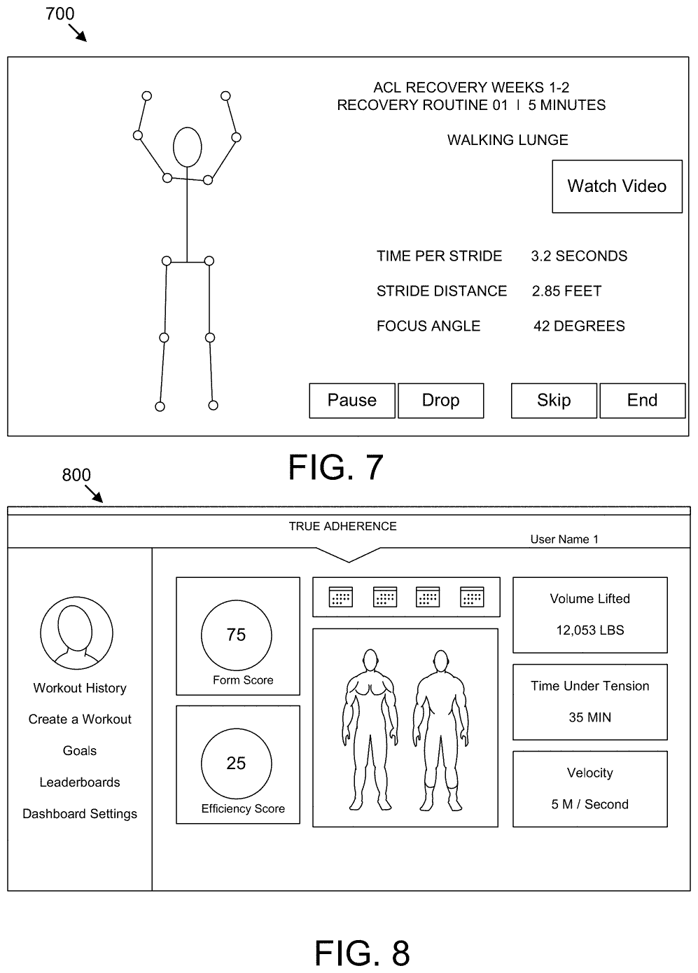

FIG. 7 shows a schematic diagram of an interface 700 of a rehabilitation assessment tool 270 associated with the platform. Using angle measurement, therapists are able to determine the angles of mobility for patients post-surgery or injury. This replaces the use of traditional goniometers that have a significant margin of error. Mobility and rehabilitation exercise information can be carried over in the system to continue to evaluate the patient once they have been dismissed from therapy to continue the healing and strengthening process.

FIG. 8 depicts another schematic diagram of an interface 800 that users have access to for their data. This may be accessed via a web portal or phone application or another general computing device. Through this portal users may change goal related inputs to recalibrate the suggestions of the system.

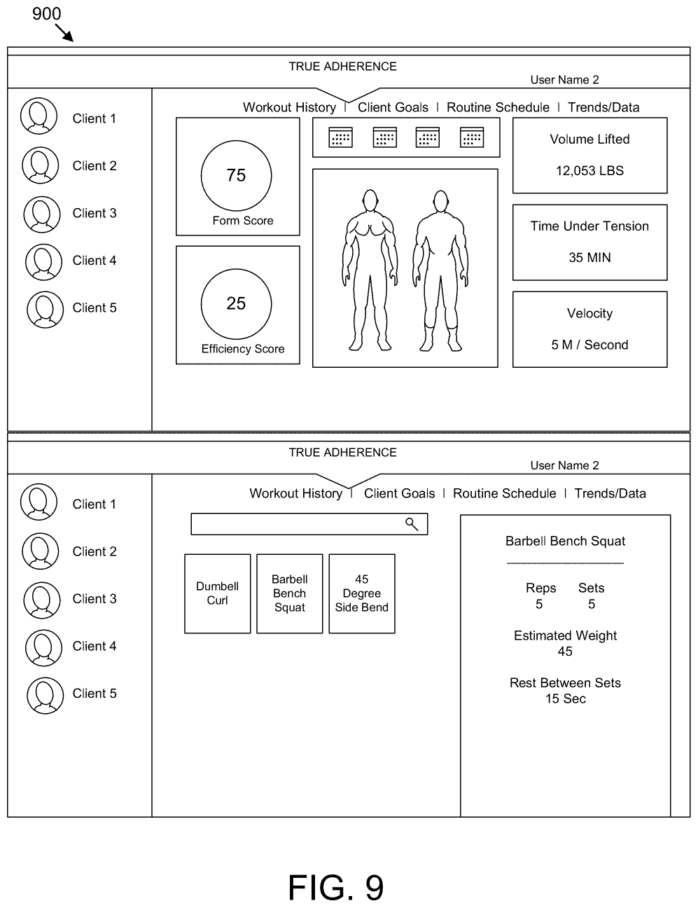

FIG. 9 shows a schematic diagram of a professional portal interface 900 where therapists or personal trainers may access the data of clients with permission. Professionals may recalibrate the output of the system, override workout plans with their own, and provide additional insight on client performance based on observation. In some embodiments, the system 100 will prompt input from the professional trainer. The professional trainer will, in some embodiments adjust, recalibrate, override, or otherwise manipulate the adjusted workout for a user.

To properly calibrate the system, various physical assessments are conducted by the different types of platform module. For the most part User Assessments include, but are not limited to:

1. Overhead squat (unloaded)

2. Standing assessment (Facing Camera)

3. Standing Assessment (Side View)

4. Single leg Squat

5. Arm extension, rotation, raise and front lift

6. 1-mile timed

7. Shoulder mobility

8. Two-Hand bar hold stepover

9. Pushing assessment

10. Pulling assessment

11. Rockport Walk Test

12. YMCA 3-Minute Step Test

13. Y-Balance Reach

14. Davies Test

15. Shark Test

These assessments identify the mobility, flexibility, cardiovascular and resistance training constraints of the client. The gathered information guides the decision engine 135 to make recommendations according to those constraints. The system aims to continually evaluate the capability of the client as they complete the recommended programming.

If a user begins to perform poorly during any given workout, the system will reevaluate the user sooner than the typical once a month assessment. This is in case the user has an unidentified injury. In some cases, behavioral questions will be asked during the workout to gauge mental and emotional factors that cannot be measured physically. Answers to those questions may lead to a modification of the workout to ensure optimal training for that session.

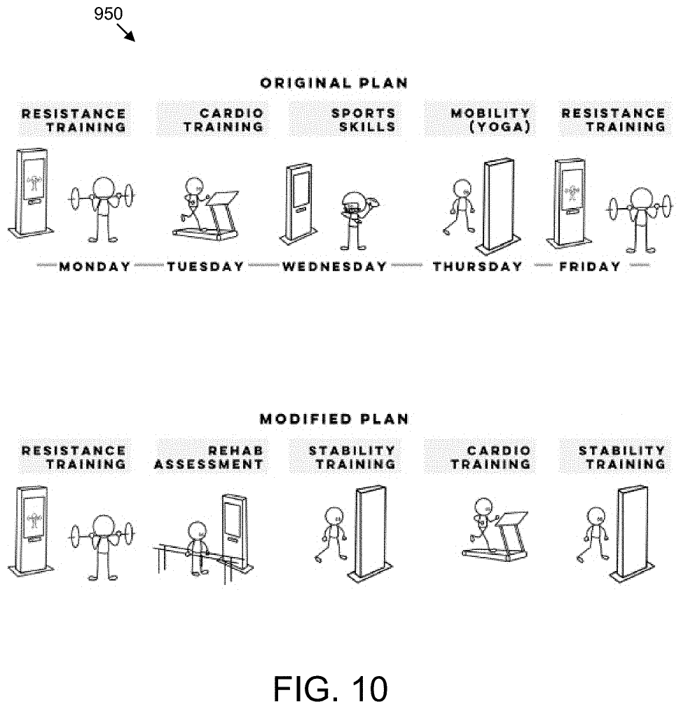

FIG. 10 illustrates how the system modifies workouts based on observed information from the various inputs. In this example, an athlete went from sport specific programming to a focus on rehabilitation and mobility after a new rehabilitation assessment was performed.

Key variables determining workout variation are determined based on functional performance metrics the system observes across the different activities. All of this information is added to the user profile. The key factors, of various activities, are listed and described below:

Resistance Training Variables:

Form Score:

The form score quantifies movement conformity to a reference movement profile. Movements may be characterized by three dimensional positions or deviations relative to the profile. In some embodiments, one or more sensors may detect deviations of individual position nodes (e.g., joints) relative to a target position. In some embodiments, the sensors detect deviations of an aggregate subset of nodes relative to the total number of nodes. In some embodiments, the sensors detect movements or deviations from expected eccentric or concentric paths, such as translational deviations, stutters, or discontinuities. In some embodiments, the sensors detect timing characteristics (e.g., pauses, transitions, etc.) of portions or entire movements, which may be equated in time or frame captures or an equivalent indicator for the passage of time. Deviations outside of tolerance bounds, or within different tolerance ranges, might be used to quantitatively impact a scoring metric through algorithm that considers and weights one or more positional or timing variables. In some embodiments, the reference movement profile is based on the original movement assessment for the user and/or a standardized movement profile. In some embodiments, tolerance bounds are created for an individual's movement relative to the individual's reference movement profile. For example, a person who has tight calves and hips might only be able to go to a 120° knee flexion during a squat movement. During a workout, it might be expected that the individual should hit a 115° to 122.5° knee flexion. If the person performs under this (e.g., 125° of flexion under load) their score is influenced by some quantity or percentage out of the upper bound tolerance. Likewise, if the user breaches the lower bound tolerance, an opposite (and perhaps different magnitude) impact may be applied to the score. Alternatively, if a user breaches the lower bound, the impact may be omitted from the Form Score calculation, depending on the potential of the movement as a condition of, or predictor for, a change in performance. However, in some embodiments, the movement is flagged to be reviewed to change the future tolerance of the exercise to the new tolerance point, and the type manner in which the adjustment to the tolerance point happens over time (e.g., abruptly, as a percentage change, or through a hysteresis algorithm) may vary in different embodiments. This example of knee flexion is only one example. In other embodiments, similar types of detection, analysis, and feedback may be performed on all of the joints for the lift during all frames where the user is under tension. (Note: Tension is defined as being engaged in eccentric/transition/concentric movement.) Continuing the squat example, tension occurs when a person goes from standing up straight to the beginning of hip hinging and knee flexion. Tension is stopped when the user returns to the starting standing position. Throughout all tension frames, the various joints are observed for adhering to proper kinematic movement patterns. Under the squat examples, knees should hit proper flexion, however, the kneecaps should track over the feet, if the knees move inward (Valgus) or outward (Varus) the kinematic tolerances are being breached. For every frame or discrete time period this persists, an error is logged by the system. These logged errors are compiled across some or all of the joint points for the total tension framed. In some embodiments, this information is used to create the Base Form Score indicative of a qualitative or quantitative assessment of correctness, conformity, stress, potential injury, or any other similar performance evaluation.

In some embodiments, the Form score from each rep is averaged or otherwise mathematically combined over all of the reps for a set. This gives the form score for a set.

In some embodiments, for a given workout, the average, or another mathematical combination, of the set scores are taken to give the exercise form score for that workout.

The average or another mathematical calculation of all the exercise scores in a workout give the workout form score for the workout.

In some embodiments, the user is assigned an ongoing form score that is equal to the form score of a particular workout (e.g., the user's last workout) or a combination of workouts (e.g., an average or chronologically weighted average over a historical period of workouts or days).

Velocity:

Velocity (meters per second) may be observed during the eccentric to transition phase (Eccentric Velocity) and transition to concentric phase (Concentric Velocity) of a lift. Although both velocities are observed, in some embodiments users are able to see both velocities, while in other embodiments users might only see a single velocity (e.g., the Concentric Velocity) as an output.

To track distance over time, in some embodiments the system tracks the joints closest to the load (e.g., squat=mid shoulder joint, curls=hands, front delt raise=hands, etc.). In other embodiments, the system may assign variable weights to different joints, for example, by assigning higher weighting factors to the joints closest to the load or, in another example, to the joints most likely to be indicative of deviations, failure, progress, or injury relative to a particular exercise.

In some embodiments, the distance used is from the starting point of a phase to the end of a phase and is dependent on the lift (e.g., Squat Eccentric Velocity Distance=Central Shoulder Joint at Neutral Standing Position to Central Shoulder Joint at bottom of squat [Neutral Position to Transition Phase]. Squat Concentric Velocity Distance=Central Shoulder Joint at bottom of squat to Central Shoulder Joint at Final Standing Position.) The time it takes for an individual to go from one point to the other may be the time used for the velocity calculation.

Time Under Tension:

Is the total time a repetition is spent in any of the Eccentric, Transition, or Concentric phases (or a combination of the phases) of a dynamic exercise (meaning the movement of joints is involved in the lift). Standing in a starting position (e.g., dumbbells in hand, barbell on shoulders, etc.) is only considered tension for static movements (e.g., loaded stand, dumbbell hold, etc.).

Loading Phases (Eccentric, Transition, Concentric)

Volume

Reps×weight

Active Time

The platform or system is able to accurately calculate the “Active Time” of a user. Active Time is defined as being under load or doing some form of active cardio in-between resistance-based sets. For example, a user does 10 reps of overhead press. Upon racking the weights (putting the weights down) the user immediately begins doing jumping jacks for 60-seconds. After the 60-seconds the user begins the next set of overhead press. Through our system, we can track when a user is moving or at rest based on observing the various joints. When lifting, angles of joints determine time under tension. When doing active rest cardio, the system is able to measure the variance in movement between joints and at what velocity and frequency that occurs. For instance, the system observes the hands during jumping jacks. If the hand is observed to stop moving, remaining at the users sides, active time will cancel or pause as long as the rest of the body is not moving rapidly (e.g., the legs aren't observed to be jogging in place based on velocities of the feet and knees). In some embodiments, Active time is used to calculate the Active Time Ratio as [Active Time During a Workout Period/Total Time Per Workout Period.] So, if a user is active for 100 seconds out of a 120 second workout their Active Time Ratio is 83.3%.

Isolated Loading/Muscular Balance:

During resistance movements that allow for independent limb movement (e.g., dumbbell curls, dumbbell overhead press, double kettlebell deadlift, Russian get-ups, etc.) the system is able to identify imbalances based on observed range of motion and differences in form score for different musculoskeletal structures. If the imbalance is measured to surpass a threshold (e.g., 2%, 5%, 10%, or another percentage between 2-10% of either form score relative to the user's reference form score, or as a difference between the form scores, the user may be instructed to do an assessment exercise (e.g., an unloaded assessment (for movements done with load) or a combined movement (e.g., dumbbell curl would be tasked with a barbell curl) to see if the issue persists with the combined movement) to further assess the user's performance.

Rate of Perceived Exertion (Input from User)

At times, the user is asked certain questions to determine the “Rate of Perceived Exertion.” This would be triggered when a user does not perform according to estimates. For instance, if the user is tasked with adding 5 Lbs to their squat and the system observes a +/−5% change in velocity and/or a +/−10% change in time under tension across a set as compared to the previously completed same workout they will be asked initial behavioral questions with multiple choice answers that may be answered via a user input on the system or via voice inputs:

“How was that set?” 1) Too hard, 2) Just Right, 3) Too Easy

“Too Hard” modifies the variables of the routine to return expected velocity and tension to normal. Variables such as longer rest time, lighter weight, or fewer reps may be introduced.

“Just Right” will make no changes, but the variable tolerances will continue to be observed on the next set. If the same +/−changes persist, the user will be prompted to modify the next set of the routine with one of the suggestions listed under the other two options.

“Too Easy” will result in a suggested increase in load, decrease in rest time or increase in reps. This is dependent on their goals and how well they outperformed the estimated performance tolerance.

Although specific percentages are referenced in some embodiments, other threshold amounts, percentages, or other indicators may be established for the change in velocity, change in time, or any other quantitative or qualitative change.

Other verbal or behavioral inquiries also may be asked of the user. For example, such questions or observations may include, but are not limited to:

Tracking a change in the time the user engages with the system (e.g., a morning workout person engages the system for an evening workout, or vice versa).

Increased Heart Rate Variability outside tolerances observed during fitness assessments.

Irregular pauses during lifts or movements.

Acknowledgement of words or other voice expressions by the user indicating they are challenged by the workout at hand or, alternatively, at ease during the workout.

In some embodiments, the system will engage the behavioral question or observation protocols to additionally provide user feedback with expressions, visualization, or other communications directed at supporting the user with a feeling of mental and emotional safety.

Cardiovascular Training Variables:

Efficiency Score:

Similar to the “Form Score” users' continuous or substantially continuous motion is evaluated throughout a workout to determine how efficient the movement patterns were throughout the duration of the exercise. The level of continuous movement may be determined by meeting a minimum frequency of movement and/or a minimum magnitude of movement. The basis of efficiency comes from an initial cardio exercise assessment such as the Rockport Walk Test or a 5-minute variable exercise on the machine or another assessment of some or all of the typical cardio indicators such as heart rate, blood oxygen levels, etc. In some embodiments, system observes the patterns of movement during the initial exercise and then, from there, the system is calibrated to adjust for any unique biomechanical needs of the user. From there, kinematic tolerances are set to determine where the user should be performing during cardio.

During a cardio workout, the optimal or programmed kinematics are applied to each step or rotation (depending on the type of cardio machine being used such as a treadmill, bike, rower). Each frame that is outside of the tolerances may be flagged as an error. For example, during the initial assessment, an ideal stride path, frequency, and length (or other indicators) are determined for a user at 2 MPH, 4 MPH, and 6 MPH, the intervals they will be doing cardio at during upcoming workouts. When the user hits 6 MPH, the stride path is slightly smaller (by 5%) the system records this for every stride that this occurs. The user is prompted to attempt to correct the issue with cues from the system. If the issue persists, future workouts are modified to work toward the perfect or prescribed from.

This serves as a unique measurement of activity and determines fatigue and injury prevention for individuals.

The individual step efficiency score may be calculated as a percentage deviation from an Optimal Path. The overall efficiency is the average (or other mathematically combined result) of each step throughout the workout. For convenience or further analysis, the efficiency score may be broken out throughout the duration of the workout for different speeds, time segments (e.g., every five minutes), and different resistances. In some embodiments, the system highlights areas where steps are routinely out of optimal or programmed path surpassing a threshold (e.g., any point above 1-10%).

The following cardio variables allow the system to coach the user as cue points to be more efficient. Other embodiments may utilize additional cardio variables as cue points. These may include, but are not limited to, Repetitions per Minute; Movement Path; Various Angles (Ankle, Knee, Elbow, Hip, Neck); Trunk Alignment; Transverse rotation; Mobility Variables: Time of Stretch; Distance of stretch; Pre-Workout Range of Motion; Post-Routine Range of Motion; Ability to Hold Movement Over Time; Interdependencies of data.

In one embodiment, the system uses the data from the various workout types (resistance, cardio, mobility, etc.) to modify future workouts and/or create alternative user profiles based on observed performance data and calculated performance data (derived directly or indirectly from the observed performance data). Therefore, a cardiovascular workout may have implications on mobility training, or a resistance training session may alter future cardio efforts. For example, during a workout if a user experiences a significant Efficiency Score drop during the final 10 minutes of a workout session primarily due to an imbalance on the right leg that causes the knee to gravitate away from the body during the forward motion. In one example, this scenario might trigger a modification to ongoing or subsequent resistance training to incorporate inner leg activation during the warmup phase and at the end of a lower body resistance session.

All of the modules work together to create the complete profile of the athlete. Through the web portal or phone application, users will be able to schedule workouts in accordance with their workout program on the platform. This enables users to utilize the most optimal version of the platform for a given workout. The reservation system will adapt programming for the client if a required module is not available where they work out (e.g., no cardio module would lead to a body-weight cardio session in front of a resistance module instead.)

Ancillary technologies may be used to inform the decision engine 135. This includes heart rate monitors, nutrition trackers, sleep monitors, step counters, strain measurement devices and other devices that track physical activity away from the platform. This supplemental data will allow for the adjustment of programming to consider performance outside of the platform (e.g., a user runs a marathon, competes in an athletic event, hikes, etc.).

FIG. 11 illustrates one embodiment of a system 100 according to one or embodiments of the disclosure including a performance assessment system. The illustrated system 100 includes a plurality of sensors 122 capable of detecting body movement and/or calculating performance metrics for a user during exercise or movement. The sensors 122 transmit data such as resistance training data, cardio data, and mobility data to a performance engine 133, which is implemented through software stored and executed on a computerized device. Further part or all of the software may be on one or more non-transitory electronic storage devices for execution on a computer processor (or multiple computer processors) in order to perform the computational assessment and user interface functions described herein. Such user interface functions additionally may be implemented using any form of hardware capable of conveying information to the user, including visual displays, touch screens, audio speakers, visual light indicators, wired and wireless platforms, and so forth.

Sensors 122 may include sensors that are capable of tracking movement of parts of the body in a three-dimensional space. The sensors 122 may also be heart rate monitors, breathing monitors, blood pressure monitors, and other sensors capable of determining physiological data of the user while they are performing exercises. In addition, the sensors 122 may include timers that track the time during and between exercises.

In some embodiments, the data transmitted from the sensors 122 to the performance engine 133 include both observed data 302 and calculated data 304. Observed data 302 refers to data natively collected by the sensors corresponding to the movements or characteristics of the user (and potentially the user's environment). Calculated data 304 includes data that is derived, in whole or in part, from the native data of the sensors. In alternative embodiments, the calculated data may be generated, at least partially, by the performance engine 133 or related electronic components. The data may be related to resistance training data 310, cardio training data 320, mobility data 330, or other performance data 340.

The performance engine 133 includes, in some embodiments, a plurality of modules configured to implement electronic instructions to gather, assess, manipulate, and communicate data. In some embodiments, the performance engine 133 includes a workout monitoring module 380 to monitor, or assist in monitoring, the motions and characteristics of the user during a workout. In some embodiments, the performance engine 133 includes a workout logging module 370 which creates a log of the data collected and/or calculated about the user's workout. In some embodiments, the performance engine 133 includes a workout planning module 360 which modifies an existing workout or create a new workout that is influenced by the metrics gathered during the user's prior workout(s). As noted herein, the workout planning module 360 may cross-reference different types of data sets in order to generate new or modified plans or profiles 402 for the user.

In some embodiments, the performance engine 133 includes an injury or deviation identification module 350 which assists with identifying potential user injuries or deviations from expected movements or workout plans. In further embodiments, the injury or deviation identification module may implement artificial intelligence, machine learning, or other constructive processing methodologies to propose and/or generate new frameworks or assessments or characteristics or metrics that, over time, may be associated with general or specific conditions that might be indicative of injuries or other positive or negative deviations from historical or expected performance. All these modules may run associated algorithms to perform steps of the methods described herein.

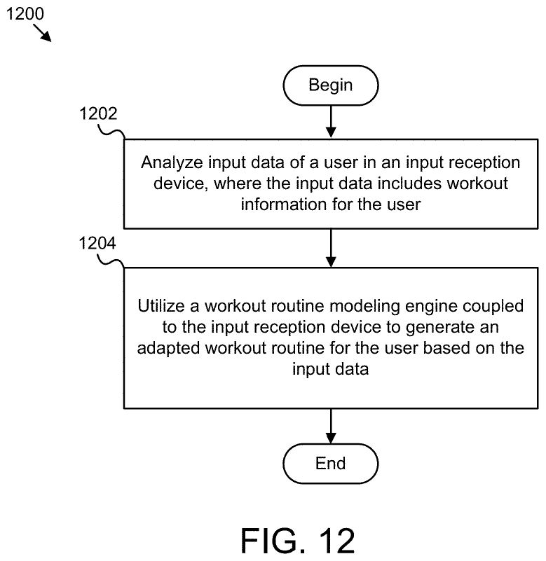

Referring to FIG. 12, a method 1200 is disclosed. At block 1202, the method 1200 includes receiving input data of a user in an input reception device, wherein the input data comprises workout information for the user. At block 1204, the method 1200 includes utilizing a workout routine modeling engine coupled to the input reception device to generate an adapted workout routine for the user based on the input data. The method 1200 then ends.

In some embodiments, the method further includes monitoring a user in at least one workout routine with the input reception device. In some embodiments, the method further includes receiving the input data through manual input by the user.

In some embodiments, the workout routine modeling engine utilizes an AI algorithm that is configured to analyze the input data and determine the adapted workout routine for the user based on the input data.

In some embodiments, the method further includes developing a reference movement profile, wherein the reference movement profile comprises expected three-dimensional positions and movements associated with the user.

In some embodiments, the method further includes determining whether the input data includes deviations from the reference movement profile. In some embodiments, the method further includes deviating from the adapted workout routine for the user when the deviations are above a threshold.

In some embodiments, the method further includes updating the reference movement profile with the deviations for subsequent workouts. In some embodiments, the method further includes developing a form score, wherein the form score quantifies conformity of the input data to the reference movement profile. In some embodiments, the method further includes averaging a form score of each repetition of an exercise to determine a form score of a set of repetitions.

In some embodiments, the input data is manually input by a user. For example, the user may input how they felt during a workout, how many repetitions were performed, weights used, time duration. All the information may be input by the user in some embodiments.

In some embodiments, the adapted routine may be approved of by a human trainer. The human trainer may be prompted to review certain aspects and confirm the decision made by AI or other algorithms about adjustments to a workout plan.

In the context of the present application, where embodiments of the present invention constitute a method, it should be understood that such a method is a process for execution by a computer, i.e. is a computer-implementable method. The steps of the method therefore reflect various parts of a computer program, e.g., parts of one or more algorithms.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, an embedded system, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a storage class memory (SCM), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages such as MatLab or Python. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions such as application specific integrated circuits (ASICs) or other hardware described herein.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the above description, specific details of various embodiments are provided. However, some embodiments may be practiced with less than all of these specific details. In other instances, certain methods, procedures, components, structures, and/or functions are described in no more detail than to enable the various embodiments of the invention, for the sake of brevity and clarity.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A system to generate an adapted workout plan, the system comprising:
an input reception device to receive input data from a user; and
a workout routine modeling engine coupled to the input reception device, the workout routine modeling engine configured to:
analyze the input data from a user; and
generate an adapted workout routine for the user based on the input data.

2. The system of claim 1, wherein the input reception device includes a camera to record a workout routine of the user.

3. The system of claim 2, wherein the workout routine modeling engine is configured to analyze parameters of the workout routine of the use.

4. The system of claim 1, wherein the input reception device is configured to receive manually entered data from the user.

5. The system of claim 1, wherein the workout routine modeling engine includes an AI algorithm that is configured to analyze the input data.

6. The system of claim 1, wherein the workout routine modeling engine develops a reference movement profile, wherein the reference movement profile comprises expected three-dimensional positions and movements associated with the user.

7. The system of claim 6, wherein the workout routine modeling engine is configured to determine whether the input data includes deviations from the reference movement profile.

8. The system of claim 7, wherein the workout routine modeling engine is configured to deviate from the adapted workout routine for the user when the deviations are above a threshold.

9. The system of claim 8, wherein the workout routine modeling engine is configured to update the reference movement profile with the deviations for subsequent workouts.

10. The system of claim 9, wherein the workout routine modeling engine is further configured to develop a form score, wherein the form score quantifies conformity of the input data to the reference movement profile.

11. The system of claim 10, wherein the workout routine modeling engine is configured to average a form score of each repetition of an exercise to determine a form score of a set of repetitions.

12. A method for generating an adapted workout plan, the method comprising:
analyzing input data of a user received by an input reception device, wherein the input data comprises workout information for the user; and
utilizing a workout routine modeling engine coupled to the input reception device to generate an adapted workout routine for the user based on the input data.

13. The method of claim 12, further comprising monitoring a user in at least one workout routine with the input reception device.

14. The method of claim 12, further comprising receiving the input data through manual input by the user.

15. The method of claim 12, wherein the workout routine modeling engine utilizes an AI algorithm that is configured to analyze the input data and determine the adapted workout routine for the user based on the input data.

16. The method of claim 12, further comprising developing a reference movement profile, wherein the reference movement profile comprises expected three-dimensional positions and movements associated with the user.

17. The method of claim 16, further comprising determining whether the input data includes deviations from the reference movement profile.

18. The method of claim 17, further comprising deviating from the adapted workout routine for the user when the deviations are above a threshold.

19. The method of claim 18, further comprising updating the reference movement profile with the deviations for subsequent workouts.

20. The method of claim 12, further comprising:
developing a form score, wherein the form score quantifies conformity of the input data to the reference movement profile; and
averaging a form score of each repetition of an exercise to determine a form score of a set of repetitions.
